(19) United States
(12) Patent Application Publication
(10) Pub. No.: US 2022/0024986 A1
(43) Pub. Date: Jan. 27, 2022

(54) MOLECULAR SIGNATURE GENERATION

(71) Applicant: OpenNano, Inc., Palo Alto, NJ (US)

(72) Inventors: Kim E. Drexler, Oxford (GB); Serge Faguet, Moscow (RO); Ashwin Gopinath, Palo Alto, CA (US); Vadim Pinskiy, Fair Lawn, NJ (US); Tj Brunette, Seattle, WA (US); George Markou, Foster City, CA (US)

(21) Appl. No.: 17/342,938

(22) Filed: Jun. 9, 2021

Related U.S. Application Data
(60) Provisional application No. 63/036,627, filed on Jun. 9, 2020.
(60) Provisional application No. 63/067,045, filed on Aug. 18, 2020.

(51) Int. Cl. C07K 14/00; C07K 19/00; C12Q 1/6876

(52) U.S. Cl. CPC C07K 14/001; C07K 19/00; G06N 3/02; C12Q 2563/185; C07K 2319/40; C12Q 1/6876

(57) ABSTRACT

The present invention provides bi-stable functionalized organic molecule and methods of utilizing these bi-stable functionalized organic molecule.

### CROSS-REFERENCE TO RELATED APPLICATION

This application claims benefit of U.S. Provisional Patent Application No. 63/036,627, which was filed in the U.S. Patent and Trademark Office on Jun. 9, 2020, and U.S. Provisional Patent Application No. 63/067,045, which was filed in the U.S. Patent and Trademark Office on Aug. 18, 2020, the entire contents of which are incorporated herein by reference for all purposes.

### FIELD OF THE INVENTION

The present disclosure generally relates to molecular signature generation using a bi-stable functionalized organic molecule (functionalized gadget), and methods of utilizing the bi-stable functionalized organic molecule in the determination of proteins, small molecules, metabolites, or a combination thereof in a fluidic sample.

### BACKGROUND OF THE INVENTION

Molecular quantification is the cornerstone of almost all bio-chemical studies like diagnosis and drug-discovery. Over the last few decades, an assortment of molecular quantification methods have been developed, finding extensive use in laboratory and clinical settings. The various underlying technologies, including mass-spectrometry, ELISA, and UV-absorption, have improved tremendously in sensitivity and throughput over the last two decades due to improvement in automation and micro-fabrication. However, the ability to quantify nucleic acids (DNA and RNA) has advanced in leaps and bounds compared to other classes of molecules. It is now possible to simultaneously quantify millions of different DNA (or RNA) strands at single-molecule resolution rapidly (few hours) and economically (<$100). This level of throughput and sensitivity is primarily due to the ability to amplify nucleic acids using PCR, which is a capability not currently reproducible with any other class of molecules.

One of the consequences of this extraordinary capability is that biotechnology, and healthcare as a whole, has become heavily genome-centric, overtly focusing on quantifying the genes. While such an approach has proven to be useful in several situations, nucleic acids (DNA and RNA) do not provide a complete picture. This is due to the fact that genes are merely “blueprints” about the likelihood of developing a characteristic. These “blueprints” must first be transcribed into RNA. Then, the RNA must be translated into proteins that work with small molecules. Proteins are the real “actors” in an individual's body that work with other small molecules to modulate the functional characteristics. Thus, in addition to characterizing the genes, it is also important to precisely quantify all the proteins and small molecules within a system to build a molecularly precise snapshot of their health. Such a capability is however not currently available due to technical limitations currently associated with quantifying non-nucleic acid molecules.

The proteomics assay, as defined herein, is an experimental-computational framework designed to rapidly create a nucleic acid signature based on all the molecules present in a sample solution. This nucleic acid signature is achieved using a library of functionalized gadgets; each functionalized gadget is designed to interact with a plurality of target analytes in a given sample (such as proteins, small molecules, and metabolites in a fluidic sample for example in a subject) each having a slightly different affinity to the functionalized gadget. These functionalized gadgets also carry a unique nucleic-acid tag that is created through a panel approach (such as mRNA display for example). The assay is designed to distinguish between complexed functionalized gadgets from other molecules and analytes in solution and from uncomplexed functionalized gadgets to enable a chemical reaction network (FIG. 1). Complexed functionalized gadgets are bound to a plurality of target molecules. The downstream analysis and data models are used as the framework for the separation and subsequent assay. After separation, the assay can yield a pool of nucleic acids each of which is a marker of a single complexed functionalized gadget. Assuming a pair-wise interaction between functionalized gadgets and molecules in solution are all unique, the above assay is guaranteed to yield a unique signature for a given sample solution (FIG. 1).

What is needed is an improved functional gadget that would generate a unique signature in a given fluidic sample.

### BRIEF DESCRIPTION OF THE DRAWINGS

FIG. 1 is a graphical representation of molecular signature generation process.

FIGS. 2A-2C comprises three graphical representations of the functional gadget. FIG. 2A is a graphical representation of the functional gadget. FIG. 2B is a graphical representation showing the functional gadget in the “closed” configuration and the conformational change in the “open” configuration after contacting a molecule of interest in a fluidic sample. FIG. 2C is a graphical representation of the proposed flow-chart of the proposed signature generation assay: Step 1 is incubation with the analyte(s) of interest in a fluidic sample; Step 2 is purification by subjecting the functional gadget, in a “closed” and “open” configuration to a purification column; and Step 3 is cleavage of the barcodes and sequencing (or genotyping).

FIGS. 3A and 3B are a representation of the functionalized gadget relative to a standard molecular-weight size marker (ladder). FIGS. 3A and 3B show the opening/closing of the functionalized gadget relative to the standard ladder. This opening/closing mechanism is key to the function of the functionalized gadget. These figures are a set of standards that are used to identify the approximate size of a functionalized gadgets on a gel during electrophoresis, using the principle that molecular weight is inversely proportional to migration rate through a gel matrix.

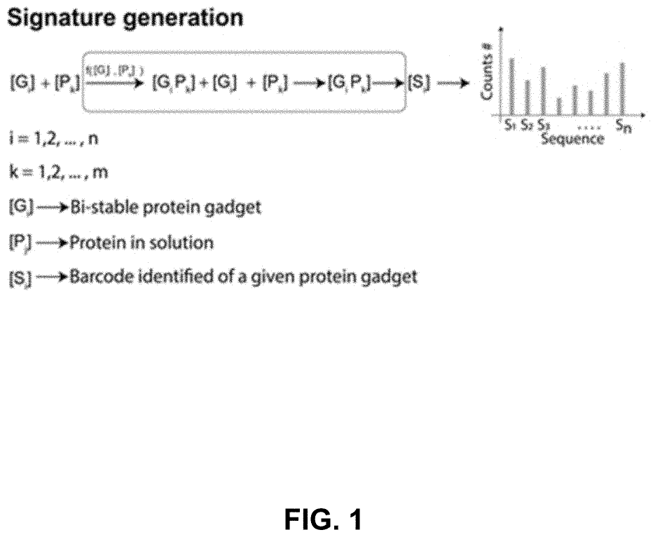

### SUMMARY OF THE INVENTION

In one aspect, disclosed herein are a bi-stable functionalized organic molecules (functionalized gadgets), the bi-stable functionalized organic molecules comprise: (a) two structurally rigid, constant regions (constant region 1 and constant region 2); (b) two modifiable, variable regions (variable region 1 and variable region 2); (c) a purification tag; and (d) a barcode region; wherein variable region 1 is covalently attached to an interior position or a terminal position of constant region 2, constant region 2 is covalently attached to an interior position or a terminal position of constant region 1, variable region 2 is covalently attached to an interior position or a terminal position of constant region 1, the purification tag is attached to an interior position of constant region 2; and the barcode is covalently attached to an interior position or a terminal position of variable region 1 or variable region 2; wherein the variable region 1 and variable region 2 interact through intramolecular hydrogen bonding, zwitterionic bonding, or a combination of intramolecular hydrogen bonding and zwitterionic bonding.

Other features and iterations of the invention are described in more detail below.

### DETAILED DESCRIPTION OF THE INVENTION

The present disclosure encompasses a bi-stable functionalized organic molecule (functionalized gadget) and methods for utilizing these molecules to determine the concentration of an analyte (such as a protein, small molecule, or metabolite) in a fluidic sample through a nucleic acid signature.

### (I) Functionalized Gadget Molecule

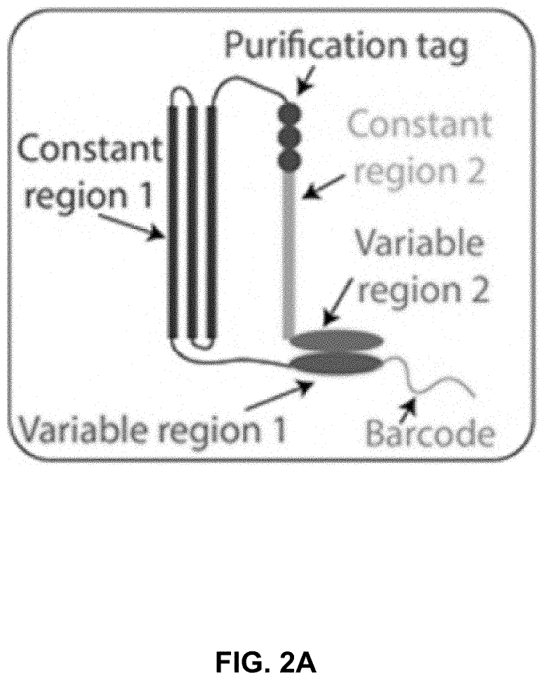

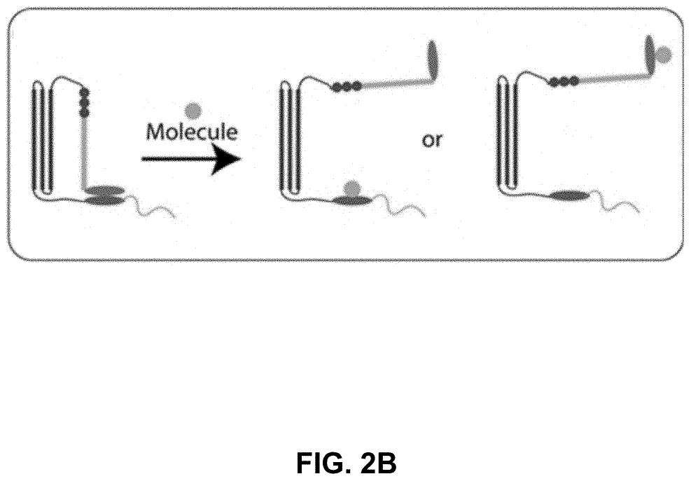

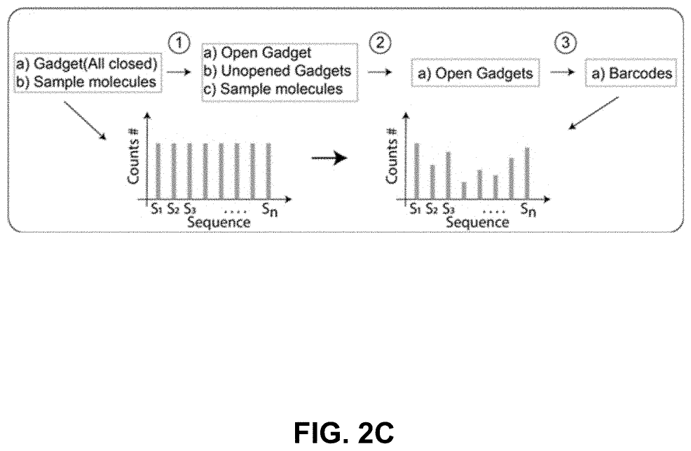

In one aspect, disclosed herein, are a bi-stable functionalized organic molecules (functionalized gadgets), the bi-stable functionalized organic molecules comprise: (a) two structurally rigid, constant regions (constant region 1 and constant region 2); (b) two modifiable, variable regions (variable region 1 and variable region 2); (c) a purification tag; and (d) a barcode region; wherein variable region 1 is covalently attached to an interior position or a terminal position of constant region 2, constant region 2 is covalently attached to an interior position or a terminal position of constant region 1, variable region 2 is covalently attached to an interior position or a terminal position of constant region 1, the purification tag is attached to an interior position of constant region 2; and the barcode is covalently attached to an interior position or a terminal position of variable region 1 or variable region 2; and wherein the variable region 1 and variable region 2 interact through intramolecular hydrogen bonding, zwitterionic bonding, or a combination of intermolecular hydrogen bonding and zwitterionic bonding. The functionalized gadget is depicted in FIG. 2A. As shown in FIG. 2A, variable region 1 and variable region 2 interact with each other through intermolecular hydrogen bonding, zwitterionic bonding, or a combination of intermolecular hydrogen bonding and zwitterionic bonding forming a complex. This complex is termed a “closed functionalized gadget.”

(a) Barcode

The functionalized gadget comprises a barcode region. The barcode region enhances the detection ability of the functionalized gadget molecule.

In various embodiments, the barcode region comprises a nucleic acid. The barcode region comprises natural nucleic acids or unnatural nucleic acids. Non-limiting examples of natural nucleic acids may be DNA, RNA, or a combination of DNA and RNA. Non-limiting examples of unnatural nucleic acids may be peptide nucleic acid (PNA), morpholino- and locked nucleic acid (LNA), glycol nucleic acid (GNA), and threose nucleic acid (TNA). In an embodiment, the barcode region comprises RNA. The mRNA display has the capability for joining the associated pipeline of functionalized gadgets to mRNA through mRNA multiplexing.

Various nucleotides comprise the nucleic acid. These nucleotides comprises natural nucleotides, unnatural or synthetic nucleotides, or a combination of natural and unnatural nucleotides. Non-limiting examples of suitable natural nucleotides comprise natural nitrogenous bases such as guanine, adenine, cytosine, thymine, and uracil. Non-limiting examples of unnatural or synthetic nucleotides are natural bases which are modified at various positions on the natural bases with a methyl, substituted methyl, ethyl, isobutyl, benzyl, halides, or cyano groups.

In some embodiments, DNA, RNA, or the combination of DNA and RNA in the barcode region may be further conjugated, replaced, or conjugated and replaced with additional structures. These additional structures would enable non-genomic detection of the barcode and can be utilized for real time detection.

In other embodiments, DNA, RNA, or the combination of DNA and RNA may further be tailored to the sample to identify a specific analyte such as proteins, small molecules, and metabolites in a fluidic sample for example or selected for additional properties. DNA or RNA can be uniquely conjugated into the individual functionalized gadget or two or more functionalized gadget through various methods known in the art.

The selection of the appropriate DNA, RNA, or a combination of the DNA and RNA depends on the analyte of interest (such as proteins, small molecules, and metabolites in a fluidic sample for example). Methods are known in the art to specifically determine the appropriate selection of the appropriate DNA, RNA, or a combination of the DNA and RNA to the specific analyte.

Generally, the stability of DNA is greater than RNA. Deoxyribose sugar in DNA is less reactive due to C—H bonds on the second carbon (C2). DNA is considered stable in alkaline conditions. In contrast, the ribose sugar in RNA is more reactive because of the presence of hydroxyl group on C2. RNA is considered not stable in alkaline conditions due to the fact the bases can easily deprotonate a hydrogen atom from the —OH on C₂.

As described above, DNA, RNA, or the combination of the DAN and RNA may comprise unnatural or synthetic nucleotides. Generally, the amount of the unnatural or synthetic nucleotides in the DNA, RNA, or the combination of the DAN and RNA may be at least 1%, at least 5%, at least 10%, at least 25%, at least 50%, at least 75%, at least 90%, at least 95%, or at least 99%.

In general, the DNA, RNA, or the combination of the DAN and RNA may comprise from about 20 base pairs of nucleotides to about 5000 base pairs of nucleotides. In various embodiments, the DNA, RNA, or the combination of the DAN and RNA may comprise from about 20 base pairs of nucleotides to about 5000 base pairs of nucleotides, from about 20 base pairs of nucleotides to about 100 base pairs of nucleotides, from about 100 base pairs of nucleotides to about 500 base pairs of nucleotides, from about 500 base pairs of nucleotides to about 1000 base pairs of nucleotides, or from about 1000 base pairs of nucleotides to about 5000 base pairs of nucleotides. The lengths of the base pairs may be approximately equal or may be different. Each length of the base pairs may be tailored to the specific analyte.

The quantification of the barcode region in the functionalized gadget molecule can be achieved through the conversion to DNA and then sequencing of DNA or genotyping of the DNA, using existing methods known in the art.

The barcode region is covalently attached to an internal or a terminal position of variable region 1 or variable region 2. In some embodiments, the barcode region is covalently attached to the terminal end of variable region 1. In other embodiments, the barcode region is covalently attached to an internal position of variable region 1. In yet other embodiment, the barcode region is covalently attached to the internal position of variable region 2. In still another embodiment, the barcode region is covalently attached to the terminal position of variable region 2. However, the terminal end of variable region 1 is optimal for biochemical formation methods and ensures that the functionalized gadget is fully conjugated with variable region 1.

Non-limiting examples of barcodes include any sequence of DNA or RNA.

(b) Variable Region 1

The functionalized gadget comprises variable region 1. Variable region 1 can be a structured set of peptides, an unstructured set off peptides, or a combination of structured and unstructured set of peptides. Variable region 1 further comprises at least one hydrophobic region.

Variable region 1 may comprise standard L-amino acids, standard D-amino acids, nonstandard unnatural amino acids, amino acid analogs, or a combination thereof. Non-limiting suitable amino acids include standard amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), non-standard unnatural amino acids (e.g., L-DOPA, GABA, 2-aminobutyric acid, and the like), amino acid analogs, or combinations thereof. Amino acid analogs include α-hydroxy analogs (e.g., methionine hydroxy analog), as well side chain protected analogs or N-derivatized amino acids.

Variable region 1 may comprise at least 1% of nonstandard unnatural amino acids, amino acid analogs, or a combination thereof. Generally, the amount of nonstandard unnatural amino acids, amino acid analogs, or a combination thereof may be at least 1%, at least 5%, at least 10%, at least 25%, at least 50%, at least 75%, at least 90%, at least 95%, or at least 99%.

In general, the number of amino acids in variable region 1 may range from about 3 amino acids to about 100 amino acids. In various embodiments, the number of amino acids in variable region 1 may range from about 3 amino acids to about 100 amino acids, from about 3 amino acids to about 10 amino acids, from about 10 amino acids to about 25 amino acids, from about 25 amino acids to about 50 amino acids, from about 50 amino acids to about 75 amino acids, and from 75 amino acids to about 100 amino acids.

(c) Constant Region 1

Constant region 1 is a structurally rigid, constant region. Constant region 1 may further comprise regions for covalent attachments. These covalent attachments can be externally triggered (such as optical, electrical, or chemically triggering in nature).

Constant region 1 comprise sequences of amino acids. Suitable amino acids may be standard L-amino acids, standard D-amino acids, unnatural amino acids, amino acid analogs, or a combination thereof. Non-limiting suitable amino acids include standard amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), non-standard unnatural amino acids (e.g., L-DOPA, GABA, 2-aminobutyric acid, and the like), amino acid analogs, or combinations thereof. Amino acid analogs include α-hydroxy analogs (e.g., methionine hydroxy analog), as well side chain protected analogs or N-derivatized amino acids.

Generally, the number of amino acids in constant region 1 may range from about 3 amino acids to about 200 amino acids. In various embodiments, the number of amino acids in constant region 1 may range from about 3 amino acids to about 200 amino acids, from about 3 amino acids to about 10 amino acids, from about 10 amino acids to about 25 amino acids, from about 25 amino acids to about 50 amino acids, from about 50 amino acids to about 75 amino acids, from 75 amino acids to about 100 amino acids, from about 100 amino acids to about 150 amino acids, or about 150 amino acids to about 200 amino acids.

Constant region 1 may comprise at least 1% of nonstandard unnatural amino acids, amino acid analogs, or a combination thereof. Generally, the amount of nonstandard unnatural amino acids, amino acid analogs, or a combination thereof in constant region 1 may be at least 1%, at least 5%, at least 10%, at least 25%, at least 50%, at least 75%, at least 90%, at least 95%, or at least 99%.

(d) Purification Tag

The functionalized gadget comprises a purification tag. The purification tag allows for detection of the “open conformation” versus the “closed conformation” functionalized gadget. The purification tag is covalently attached to an internal of position of constant region 2. The purification tag can also be utilized to separate the “open conformation” versus the “closed conformation” functionalized gadget. In various embodiments, the purification tag can be further functionalized with additional components or compounds that would allow for an easy separation of the “open conformation” versus the “closed conformation” functionalized gadget.

In general, the purification tag is a protein tag or a peptide tag. Non-limiting examples of the tags may be an ALFA-tag, a C-tag, an E-tag, a FLAG-tag, a S-tag, a T7-tag, a TC-tag, a Ty-tag, a SdyTag, a BCCP-tag, a SNAP-tag, a Nus-tag, a CRDSAT-tag, a Tista-tag, an anti-His-tag, or an anti-XX-tag.

(e) Constant Region 2

Constant region 2 is a structurally rigid, constant region. Constant region 1 may further comprise regions for covalent attachment. These covalent attachments can be externally triggered (such as optical, electrical, or chemically triggering in nature).

Constant region 2 comprise sequences of amino acids. Suitable amino acids may be L-amino acids, D-amino acids, unnatural amino acids, amino acid analogs, or a combination thereof. Non-limiting suitable amino acids include standard amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), non-standard unnatural amino acids (e.g., L-DOPA, GABA, 2-aminobutyric acid, and the like), amino acid analogs, or combinations thereof. Amino acid analogs include α-hydroxy analogs (e.g., methionine hydroxy analog), as well side chain protected analogs or N-derivatized amino acids.

Generally, the number of amino acids in constant region 2 may range from about 3 amino acids to about 200 amino acids. In various embodiments, the number of amino acids in constant region 2 may range from about 3 amino acids to about 200 amino acids, from about 3 amino acids to about 10 amino acids, from about 10 amino acids to about 25 amino acids, from about 25 amino acids to about 50 amino acids, from about 50 amino acids to about 75 amino acids, from 75 amino acids to about 100 amino acids, from about 100 amino acids to about 150 amino acids, or about 150 amino acids to about 200 amino acids.

Constant region 2 may comprise at least 1% of nonstandard unnatural amino acids, amino acid analogs, or a combination thereof. Generally, the amount of nonstandard unnatural amino acids, amino acid analogs, or a combination thereof in constant region 1 may be at least 1%, at least 5%, at least 10%, at least 25%, at least 50%, at least 75%, at least 90%, at least 95%, or at least 99%.

(f) Variable Region 2

The functionalized gadget comprises variable region 2. Variable region 2 can be a structured set of peptides, an unstructured set off peptides, or a combination of structured and unstructured set of peptides. Variable region 2 further comprises at least one hydrophobic region.

Variable region 2 may comprise standard L-amino acids, D-amino acids, non-standard unnatural amino acids, amino acid analogs, or a combination thereof. Non-limiting suitable amino acids include standard amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), non-standard unnatural amino acids (e.g., L-DOPA, GABA, 2-aminobutyric acid, and the like), amino acid analogs, or combinations thereof. Amino acid analogs include α-hydroxy analogs (e.g., methionine hydroxy analog), as well side chain protected analogs or N-derivatized amino acids.

Variable region 2 may comprise at least 1% of nonstandard unnatural amino acids, amino acid analogs, or a combination thereof. Generally, the amount of nonstandard unnatural amino acids, amino acid analogs, or a combination thereof in variable region 2 may be at least 1%, at least 5%, at least 10%, at least 25%, at least 50%, at least 75%, at least 90%, at least 95%, or at least 99%.

In general, the number of amino acids in variable region 2 may range from about 3 amino acids to about 100 amino acids. In various embodiments, the number of amino acids in variable region 1 may range from about 3 amino acids to about 100 amino acids, from about 3 amino acids to about 10 amino acids, from about 10 amino acids to about 25 amino acids, from about 25 amino acids to about 50 amino acids, from about 50 amino acids to about 75 amino acids, and from 75 amino acids to about 100 amino acids.

(g) Conformational Change in Functionalized Gadget

As shown in FIG. 2A, variable region 1 and variable region 2 interact with each other forming a complex. This intermolecular interaction comprises intermolecular hydrogen bonding, zwitterionic bonding, or a combination of intermolecular hydrogen bonding and zwitterionic bonding. Due to this intermolecular interaction, his complex is termed a “closed functionalized gadget.” The intermolecular interaction of variable region 1 and variable region 2 changes in the presence of an analyte in a fluidic sample of interest (such as a protein, a small molecule, or a metabolite for example). As the analyte of interest contacts with either variable region 1 or variable region 2, the intermolecular interaction is disrupted by releasing a portion of the intermolecular hydrogen bonding, zwitterionic bonding, or a combination of intermolecular hydrogen bonding and zwitterionic bonding. By releasing a portion of these intermolecular interaction, the purification tag becomes revealed and is termed an “open functionalized gadget.”

(h) Methods for Preparing Functionalized Gadgets

A variety of methods can be used to prepare the functionalized gadgets. Non-limiting example of these methods may be solid phase synthesis or solution phase peptide synthesis. In these methods, known nitrogen protecting groups (such as BOC, FMOC), various coupling reagents (such as carbodiimides), deprotecting reagents (such as TFA, DBU, piperidine, etc), solvents, and purification (such as crystallization, precipitation, or chromatography) may be utilized as known in the art.

### (II) A Method for Determining the Concentration of a Molecule in a Fluidic Sample.

Another aspect of the present disclosure encompasses a method for determining the concentration of an analyte of interest in a fluidic solution. The method comprises: (a) providing information from a computer algorithm; (b) providing at least functionalized gadget; (c) contacting the at least functionalized gadget with a fluidic solution comprising at least one analyte; (d) binding the at least one functionalized gadget with the at least one analyte wherein the functionalized gadget molecule undergoes a conformational change upon binding with the analyte molecule; (e) purifying the fluidic solution comprising bound functionalized gadgets with the analyte from unbound functionalized gadgets; (f) identifying the at least one bound functionalized gadgets and at least one unbound functionalized gadgets through sequencing, deep sequencing, or a combination thereof; (g) identifying a unique signature of the fluidic sample; and (h) analyzing the data obtained from the fluidic sample; wherein the information from the computer algorithm in step (a) comprises information for converting molecular binding information into nucleic acid information; information from traditional genotyping; information from a mathematical association study; or a combination thereof.

The method further comprises: (i) optimizing the position of the purification tag and the purification tag on the functionalized gadget in the method; (j) optimizing the variable region 1 and variable region 2 in the functionalized gadget; and (k) optimizing the functionalized gadget to a given fluidic sample. This method and further optimizations provide an accurate measurement of analytes of interest (such as proteins, small molecules, and metabolites) present in a fluidic sample such as phenotypical, historical, or genetic fluidic samples.

(a). Providing Information from a Computer Algorithm

The first step in the method comprises providing information from a computer algorithm. Generally, the information from the computer algorithm comprises information for converting molecular binding information into nucleic acid information, information from traditional genotyping, information from a mathematical association study, or a combination thereof. The information from the computer algorithm identifies specific nucleic acids, which are complimentary to the at least one analyte in the fluidic sample of the analyte of interest such as proteins, small molecules, or metabolites.

(b) Providing at Least One Functionalized Gadget

The next step in the method comprises providing at least one functionalized gadget. The at least one functionalized gadget is described in more detail above in Section (I). The at least one functionalized gadget is designed to bind with analytes of interest such as proteins, small molecules, metabolites, or a combination of two or more these of interest. Each functionalized gadget will bind an analyte of interest with varying binding affinities in a fluidic sample.

(c) Contacting the at Least Functionalized Gadget with a Fluidic Solution Comprising at Least One Analyte

The next step in the method comprises contacting the at least one functionalized gadgets with a fluidic solution comprising at least one analyte.

The at least one analyte comprises proteins, small molecules, metabolites, or a combination of two or more these of interest. These analytes can be from the metabolism of a subject thereof, an analyte related to a specific disease, or a primary or secondary metabolite produced by the subject thereof. Non-limiting examples of these analytes may be amino acids, proteins, alcohols, vitamins (B2 and B12), polyols, organic acids, steroids, nucleotides (e.g. inosine-5′-monophosphate and guanosine-5′-monophosphate), drugs, fragrances, flavor, dye, pigments, pesticides and food additives with applications in agriculture, industry and pharmaceuticals.

Non-limiting examples of proteins include, but limited to, DNA-binding protein SATB2 (Q9UPW6, SATB2), Fc_MOUSE (Q99LC4), Tumor necrosis factor ligand superfamily member 18 (Q9UNG2, TNFSF18), Ubiquitin-conjugating enzyme E2 B (P63146, UBE2B), Kunitz-type protease inhibitor 3 (P49223, SPINT3) Cluster 2, Acidic leucine-rich nuclear phosphoprotein 32 family member B (Q92688, ANP32B), TGF-beta receptor type-2 (P37173, TGFBR2), Fc receptor-like protein 6 (Q6DN72, FCRL6), T-cell receptor-associated transmembrane adapter 1 (Q6PIZ9, TRAT1), Transmembrane gamma-carboxyglutamic acid protein 1 (014668, PRRG1) Cluster 3, Insulin-like growth factor-binding protein 6 (P24592, IGFBP6), Neuropeptide W (Q8N729, NPW), Vitrin (Q6UXI7, VIT), Periostin (Q15063, POSTN), C—X—C motif chemokine 16 (Q9H2A7, CXCL16) Cluster 4, Myosin-binding protein C, slow-type (Q00872, MYBPC1), Interleukin enhancer-binding factor 3 (Q12906, ILF3), Translation initiation factor eIF-2B subunit alpha (Q14232, EIF2B1), Phospholipase B-like 1 (Q6P4A8, PLBD1), Synaptotagmin-11 (Q9BT88, SYT11) Cluster 5, Type 2 lactosamine alpha-2,3-sialyltransferase (Q9Y274, ST3GAL6), Bisphosphoglycerate mutase (P07738, BPGM), Redox-regulatory protein FAM213A (Q9BRX8, FAM213A), Alpha-2-HS-glycoprotein (P02765, AHSG), MAP kinase-activated protein kinase 5 (Q8IW41, MAPKAPKS) Cluster 6, Thrombospondin-2 (P35442, THBS2), IGF-like family receptor 1 (Q9H665, IGFLR1), Growth arrest-specific protein 1 (P54826, GAS1), Twisted gastrulation protein homolog 1 (Q9GZX9, TWSG1), Desmocollin-2 (Q02487, DSC2) Cluster 7, Sushi, von Willebrand factor type A, EGF and pentraxin domain-containing protein 1 (Q4LDE5, SVEP1), Pleiotrophin (P21246, PTN), Chordin-like protein 1 (Q9BU40, CHRDL1), Growth/differentiation factor 15 (Q99988, GDF15), N-terminal pro-BNP (P16860, NPPB) Cluster 8, A disintegrin and metalloproteinase with thrombospondin motifs 13 (Q76LX8, ADAMTS13), Epidermal growth factor receptor (P00533, EGFR), N-acetylglucosamine-1-phosphodiester alpha-N-acetylglucosaminidase (Q9UK23, NAGPA), Vascular endothelial growth factor receptor 2 (P35968, KDR), and Proto-oncogene tyrosine-protein kinase receptor Ret (P07949, RET).

Non-limiting examples of sterols, include, but not limited to cholesterol, ergosterol, hopanoids, hydroxysterol, phytosterol, steroids, and zoosterol.

Non-limiting examples of amino acids, include, but not limited to standard amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), non-standard unnatural amino acids (e.g., L-DOPA, GABA, 2-aminobutyric acid, and the like), amino acid analogs, or combinations thereof. Amino acid analogs include α-hydroxy analogs (e.g., methionine hydroxy analog), as well side chain protected analogs or N-derivatized amino acids.

The addition of one or more functionalized gadget may occur at the same time or may occur at different times. These functionalized gadgets may have the same or different nucleic acids and amino acid, but the same general structure (and binding). These functionalized gadgets would provide time series information and possible temporal changes in the protein expression can be characterized as part of the signature generation.

After this contact occurs, the functionalized gadgets will bind to at least analyte (one protein, at least one small molecule, at least one metabolite, or a combination of two or more of these). In some embodiments, the at least one functionalized gadget will bind with at least one protein or a mixture of proteins of interest. In other embodiments, the at least one functionalized gadget will bind with at least one small molecule or a mixture of small molecules of interest. In yet another embodiment, the at least one functionalized gadget will bind with at least one metabolite or a mixture of metabolites of interest. In still another embodiment, the at least one functionalized gadget molecule will bind with a combination of two or more proteins, two or more small molecules, and two or more metabolites of interest.

The concentration of the at least one functionalized gadget and the analyte of interest solution can and will vary. Generally, the concentration of the at least one functionalized gadget may range from 1.0 nM to about 1.0 mM. In various embodiments, the concentration of the at least one functionalized gadget may range from 1.0 nM to about 1.0 mM, or from about 2.0 nM to about 0.5 mM. Generally, the functionalized gadget is generally used in excess. The barcode, attached to the functionalized gadget molecule, is utilized to determine the concentration in solution.

The concentration of the at least one analyte may be range from about 1 pM to about 1 μM. In various embodiments, the concentration of the at least one analyte may range from about 1 pM to about 1 μM, from about 5 pM to about 5 nM, or from about 8 pM to about 1 nM.

Generally, the temperature of contacting the at least one functionalized gadget with the at least one analyte may range from about 0 C to about 40 C. In various embodiments, the temperature of contacting the at least one functionalized gadget with the at least one analyte may range from about 0° C. to about 40° C., from 10° C. to about 35° C., or from about 20° C. to about 30° C. In one embodiment, the temperature of contacting the at least one functionalized gadget with the at least one analyte may be about 25° C.

(d) Binding the at Least One Functionalized Gadget with the Analyte Wherein the Bi-Stable Functionalized Gadget Undergoes a Physical State Change Upon Binding with the Analyte

The next step in the method comprises binding the at least functionalized gadget with the at least one analyte wherein the functionalized gadget undergoes a physical state change upon binding with the analyte. As described above, the at least one functionalized gadget is in a “closed” conformation before interaction with an analyte of interest. This “closed” configuration indicates that variable region 1 and variable region 2 are complexed through intermolecular binding such as intermolecular hydrogen bonding, zwitterionic interaction, or a combination of intermolecular hydrogen and zwitterionic binding. Upon contacting with the at least one analyte, the binding affinity for variable region 1 or variable region 2 is greater for the analyte of interest than for each other. The functionalized gadget transforms from a “closed: state to an “open” state wherein variable region 1 or variable region 2 binds with analyte of interest and releasing a portion of the intermolecular hydrogen bonding, zwitterionic interaction, or a combination of intermolecular hydrogen and zwitterionic binding. As appreciated by the skilled artisan, only a portion of the functionalized gadget introduced will bind with the analyte. Upon interaction of the analyte of interest with at least one functionalized gadget, the purification tag is revealed. The remaining unbound functionalized gadgets will remain in the “closed” configuration.

(e) Purifying the Fluidic Solution Comprising Bound Functionalized Gadget with the Analyte and Unbound Functionalized Gadget

The next step in the method comprises purifying the fluidic solution comprising bound functionalized gadget with the analyte and unbound functionalized gadget. Generally, the revealed purification tag on the “open functionalized gadget” allows for easy separation of the bound functionalized gadget molecules with the analyte and unbound or “closed functionalized gadget.” Various methods are known in the art for this purification such as size-exclusion chromatography, ion exchange chromatography, fast protein chromatography, HPLC, and column chromatography.

(f) Identifying the Bound and Unbound Functionalized Gadgets

The next step in the method comprises identifying the bound and unbound functionalized gadgets. As described above, each functionalized gadget comprises a barcode. These barcode are then sequenced using various methods and are shown in FIG. 2C. Non-limiting examples of these various methods may be genotyping, normal sequencing, deep sequencing, DNA sequencing, or a combination thereof. These methods described may be utilized with other methods known in the art.

(g) Identifying a Unique Signature of the Fluidic Sample

The next step in the method identifies the unique signature of the fluidic sample. Using the sequenced barcodes, there are various methods known in the art to identify the specific sequence and thus determine the specific functionalized gadget of interest.

(h) Analyzing the Data.

The final step in the method comprises analyzing the data. The analysis of the data comprises utilizing the sequencing data (such as DNA sequencing), the unique signature of the fluidic sample, and any additional post sequencing data. Additionally, this analysis of the data may further utilize deep learning, conventional analysis, and statistical analysis for example. Once the data is analyzed, the data would then be associated with a multitude of markers such as proteins, small molecules, metabolites, or a combination thereof.

The deep learning controller can utilize any available neural network model, including and not limited to, convolutional neural networks, reinforcement learning, and other such models. The data for these models will be formed from initial baseline calibrations and correlated to other markers (phenotypical, historical, etc.) or from other population sets. The models can be made to offer direct feedback into functionalized gadget molecule optimization.

The resulting data can be formed native or passed through a single or multiple deep learning controllers or other data filters, to allow signatory information to be derived from the assay or direct correlation to known markers. In some embodiments, the data from a single analysis can be used as part of a population or as a single data point. These correlations can be used as part of a larger formed database to allow for tailored targeting or select markers or proteome wide therapies or strategies. These markers would then be related to be various phenotypical, historical or from associated assays (genetic or otherwise) such as bio markers, genetics, family history, life expectancy, morbidity, etc. and micro-labels such as smoking, and other environmental local effects that can only be detected on situational basis. The correlation of this data would be used as part of a larger formed database to allow for tailored targeting or select markers or proteome wide therapies or strategies. The analysis of this data provides a snapshot of the entire protein expression and thereby, not only provides the analyte of interest in an individual but also provides the effect of the analyte specifically related to the markers in the individual and a population as a whole.

This technique can be related to other relational databases and can be used to separate cohorts of people based on the protein expression. This technique can be used for population studies and overall separation of groups based on protein expression. No previous information on the correlation of protein to the medical record is required.

The sequenced barcode information can also be used as a feedback for additional screening on the same or different sample set. Re-screening of the same sample can be done with additional functionalized gadgets or tailored functionalized gadgets for data confirmation and for repeat screening of the same individual, over a course of time. Results of the sequencing can also be used to inform additional feedback of the state of the functionalized gadget molecule and improved modification of the interaction with native proteins and serum (e.g. those that were not filtered or otherwise removed from pre-process steps).

The functionalized gadget may be combined with a manual or semi-automated methods of altering the sequence of the variable regions based on the signature and detected noise (baseline control sample for example). The feedback can be performed by sampling available protein structures from the pre-calculated form of available functionalized gadgets and altering the format based on that. Repeating the process until reduced noise is achieved. Additionally, feedback can also be applied on the barcode region comprising nucleic acids such as DNA or RNA to reduce interference with naturally occurring DNA or RNA segments in the specific sample.

The method further comprises: (i) optimizing the position of the purification tag and the purification tag itself; (j) optimizing the variable region 1 and variable region 2; and (k) optimizing the functionalized gadget to a given fluidic sample. This method and further optimizations provide an accurate measurement of the analyte of interest in a fluidic sample such as phenotypical, historical, or genetic fluidic samples.

The location, and identity, of the purification tag is one of the crucial factors that determine the correct operation of the functionalized gadget molecule. In order to optimize the identity and location, a plurality of functionalized gadgets designs are created each with a candidate affinity binder position at a slightly different location along the constant region 2 (to either FIG. 2A or FIG. 2B). Further, the variable region 1 and 2 in these functionalized gadgets will include peptides that are known to strongly interact with each other to ensure the functionalized gadgets are in the closed conformation. All of these designs may be purified using purification methods known in the art, functionalized with the partner of the purification tag, to capture all the designs in which the purification tag is accessible. The designs, in which the purification tag is optimally hidden, when the functionalized gadgets is closed, are collected and identified by genotyping and sequencing tools like DNA chip, qPCR, deep sequencing, or a combination thereof.

A second crucial factor in the operation of the functionalized gadget is the design of the variable region 1 and variable region 2 (FIG. 2A) which are weakly interacting with each other in the absence of competing molecules. This issue is relatively straight forward having optimized the location, and identity, of the specific purification tag. Specifically, a plurality of functionalized gadgets are created in which the affinity binder is located at the optimum location to be inaccessible in the “closed” state, by the variable states are randomized. By comparing the barcodes from this functionalized gadget molecule pool, before and after, being subjected to the purification can identify the functionalized gadgets that interacted with the purification. Since the design that was optimized to ensure the inaccessibility of the purification tag during the closed state, the designs whose variable regions interact with each other are directly deduced.

Since the stability of the functionalized gadget molecule is dependent not only on the concentration of the analyte of interest in the fluidic sample but also on the identity of the different analytes, the functionalized gadget molecule pool needs to be optimized to a given sample type. This optimization is be achieved by incubating all the functionalized gadgets whose variable regions are designed to ensure the functionalized gadget molecule is in the “closed” state with a reference sample and then incubating the solution to identify the functionalized gadgets that undergo conformational change by interaction with the reference sample.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described methods without departing from the scope of the invention, it is intended that all matter contained in the above description and in the examples given below, shall be interpreted as illustrative and not in a limiting sense.

### Example 1: Preparation of Functionalized Gadgets

The following sequences of the functionalized by in vitro transcription of a synthesized gene and subsequent translation using a cell-free protein expression system. Addition of the purification tag and barcode region were encoded at the gene level such that the gadget is functionalized during the translation process:

TABLE 1
Sequences of Functionalized Gadgets

| Sequence | |
|---|---|
| Identifi- | |
| cation | |
| No. | |
| (SEQ ID | |
| NO) | Sequences |
| SEQ ID | DQEIHQHLQHLQQQLHERIKHLVQK |
| NO 1 | LHEHLQHLFKQLQQAEKRGESEEER |
| | KKRLQQFRHEIQEQLQQVHEHVQKQ |
| | IEEIRQQIKKLLQEARKRSKTEEEH |
| | ERLKELHKQIHEALEHFHQAVQHFL |
| | EAFHKAFQEVLEAIKNGESEEEAQQ |
| | RRSRLEEELRRRLTEEEKRHQQHQE |
| | EQHHAHQQG |
| SEQ ID | DEEARKILQQLREQAERAHKHVQEV |
| NO 2 | HQQFQEIQERIRHLEERNDENVEEH |
| | LKHFERQIKHLFQQIQQIFQELQEH |
| | LKEAKEHAKKNNHSPHLKEAIEQIE |
| | QQVHQLFKQIQQIVEHLKQLLHQAK |
| | QAHENPEDERRKEQKEEASRLEEEL |
| | RRRLTEHEQRQHEQHEQQKQRHQHG |
| SEQ ID | SEELQQQIQELAKKAQEVHQHIHKL |
| NO 3 | LEEFHQLLEELRHSTDSPEELKKKL |
| | EELHQQLQKVLQKAQQLFRQLHELF |
| | RRLQEAAEKENISPEQKQHQEQQLR |
| | AVEHHFRAAQRFFKHFQQLLHQLHE |
| | HQEKERSEEERKHHEKEASRLEEEL |
| | RRRLTEREQEHRQQQKQHQEHQQHG |
| SEQ ID | DEELQKRIHELAHQLQRQIHHIQHH |
| NO 4 | IHEELKQIHEEIQQHEEKGRDKEEH |
| | KQRLHQLLHQFRQHVQELLQQIREQ |
| | VQQILQHIQEKGTNEEHVKAAEQAF |
| | KHAVEQVEQAFEQQLKQFAQHVKQA |
| | EEHSDRDEEEREKHRKQAKKERSRL |
| | EEELRRRLTEERQHHHEEHHHQQQK |
| | G |
| SEQ ID | SQEIAEELQKLAQELEHHIEELVKQ |
| NO 5 | LQKQLEHLAQQAEQAKKRGESEEER |
| | EERLQRFQQHIHEQLKQFHQHVHEK |
| | IHQIQQHVRHLLEKHNESSELEHHI |
| | QELLQQFHEHFHQALQHFQEEVQKQ |
| | LEEARRDKKSPEQRQEERSRLEEEL |
| | RRRLTEHKKAQKKHRHAQQQRG |
| SEQ ID | SQEILHELQKKFHHVQEQIRQLAKE |
| NO 6 | IQQILHEAQQLLHSDRSEEEIEEIH |
| | RQLKELLKQVHQQLRKIEQLVRQIE |
| | KQIQELRERGTHSEEQHEHIEQLLQ |
| | ELQKALKHLQEHVRKAHEQFQKILE |
| | ALQAARRNETDPEEKHKQHREASRL |
| | EEELRRRLTEEEKQQQEQHQKAQQH |
| | G |
| SEQ ID | DEELHQAIQELQQQLHKQIQKLAQQ |
| NO 7 | IHKQLHEIAQEIHELAERGRDEEEI |
| | KRRLKELLEHFEQHVREILQEIQQQ |
| | AQRILHKIRASGESEEQVQAAHHHI |
| | QRAVEHVKKHFHKQLEQFRKHVEQH |
| | QKEEEDSNTSKEEEHKRQKQHQQEE |
| | SRLEEELRRRLTEDEKRHHEHHQQQ |
| | HQQG |
| SEQ ID | DEEEIHEIQHRLEQLIHELAKHFRH |
| NO 8 | FAHHFHEVQQQLNKDTSEEQLKQIE |
| | EQLQEHHKQLEKHFRQAQKEFRKIQ |
| | QHLQQLRESEQLRKIHKQVQHALEH |
| | AEHVFEQIRQIVEQFEEQLHAAKRG |
| | KHDEEKAKKQHEERSRLEEELRRRL |
| | TEEEQQQQQHQHEHQRHG |
| SEQ ID | DQELLHQLQEKVHQVQQQIRELAQQ |
| NO 9 | IERLLQRIHKLLQSDRSEEEIQHIA |
| | QQLREFLQQVHKQLQEIAEQVEKIA |
| | QEAEEIKKRGVDEEQRKAIQQLVEH |
| | LHKHLERLQKHVHQAHEAFERILKQ |
| | LEHREKKGKPSEDHEEHHEEESRLE |
| | EELRRRLTEEEKQAQKQHQQHQRQG |
| SEQ ID | SKEELQELRQQAHQHQKQLHELLHK |
| NO 10 | IHEQLHQHAHEIKDKDPHHLQRIQQ |
| | HLQELLEHIHHIVREFQQHVKEVAQ |
| | QLREAQEKEKDEEEQRRLEEHLQHL |
| | QHALKRFKQHFEQAHEHVQQLQHAE |
| | KQSEEEHKKQHEERSRLEEELRRRL |
| | TEHKQHHEEHERRAQQHG |
| SEQ ID | DHELKEKIHHLQQQLRERIEELHRQ |
| NO 11 | IAEQLEQIAQQIHSDDGSEEEAAKR |
| | LQHLLHQFQEHVKQILQEIQQHIHE |
| | ILHQIKASGTDEHQVQQAEEHFEQA |
| | VRQVERHFQQQLKRFEEHVQRAKEE |
| | REKNRSEEEKEQAKQHEQEKHSRLE |
| | EELRRRLTEEKKKQHQHHQQHRKQG |
| SEQ ID | DHEQVHQLQQQAHEIREEIHQQLKR |
| NO 12 | IAQKIREVQQHVEEHKKHPETEEQR |
| | QHLEQLLRELHHQLERLKQHVKQVE |
| | KQFQEIQKHETDEEHLRELQEALKQ |
| | IQEAVHHLEHAIHRAQQHIKDKHSE |
| | EEHQREKSRLEEELRRRLTEHEQQQ |
| | EQAKKAHRQHQHHG |
| SEQ ID | SQEELQALQQQAQHHARKIQEKLEE |
| NO 13 | IHERLRQFAEQIKDKDPHELHKIIQ |
| | QLQELLEELQRQVEEFEKHVRHVAR |
| | QLKKHAKEEKDEERRQQLEKHLKQL |
| | QKHLKQAQEEFHQHEHLIRQLIAQL |
| | QSEEDNPEEEHEHSRLEEELRRRLT |
| | EHKQQHQRHAEQHQQQHRHG |
| SEQ ID | SHEEVHQLHREAHQIQRQIQEQLHQ |
| NO 14 | IHQHIHEVHQAVKKAQESEDTEEAQ |
| | QHLQQLLKQLQEQLERLKHHVQKVE |
| | QAFHRIQRHLEEHERKINGSEEHRK |
| | QLQKHLKHLREALEQIKRHVKQLQE |
| | AIQRQEEHIKKAKDDSSEEERHQEQ |
| | QEESRLEEELRRRLTEEREQHEQQH |
| | HQAHKEHKHG |
| SEQ ID | DEELHKAIKKLHEQAQQVHRAIHEF |
| NO 15 | QKQIEELLEQLQKKEPSEELLQELK |
| | QLEKQLHEILQQAQRLFEQLHKLFQ |
| | ELEEEAKKRPESEEHQQAAQQRREA |
| | LEEVRKHFEHVQKAFQHFQHVLQHL |
| | QHQLRHNDGNEEQQKRESRLEEELR |
| | RRLTERKQRHHRHHEQHEQQHQHG |
| SEQ ID | SEEIHHILQRLIEQLAKQIKELVEQ |
| NO 16 | LQEQLRHLQQRAQKAEERGKSEEEK |
| | QQQLEEFHKQLEKALERVHQRVAQQ |
| | IQHIQQHIAQIVKDPELRKHIEHLL |
| | EAFKQHVEQFLEEFHQRIRHHLQHP |
| | ETSDEEESRLEEELRRRLTEKEQQQ |
| | QHHQEEARKQQKKG |
| SEQ ID | DEELQEKIHQLQHQLHHKIQQIQEH |
| NO 17 | IRQQLHQIQEEIQQAIEKGRDEEEH |
| | IERLRQLLEHFRQQVHQLLQQIEQQ |
| | VKRILHHIKANGEDEHHVQQAQEHF |
| | EQQVKQVKEHFQAALKQFEEHVKKA |
| | IEEFKSEDSEEEKKKRHQHERSRLE |
| | EELRRRLTEHRRQQKEAAEEARQHH |
| | HEG |
| SEQ ID | DEELKQRIHELHQQAEQVHRQIHQV |
| NO 18 | FKKFHELLKELQAQLKEHGTETEEL |
| | RQKLQELHEELKRVLEEAQKLFREL |
| | HQLFRELQQQAKENNESEEEQEHQH |
| | RALQHVQEHVKQAQQFFKHFQRLLH |
| | ALQEIAKGTSEEEAKRRQQKHSRLE |
| | EELRRRLTEHKQQHEEQQQHHEQHH |
| | RQG |
| SEQ ID | DEEELRQLEHHLHQAQQELHEILQQ |
| NO 19 | IQEHLEQAAQLIKDKDPEELKKILQ |
| | KLRQLLRRLRHQVQRFEQLVKHVEE |
| | RLKEFHKREKDEERQKQLQEHLQQL |
| | QEHLRQAQEAFEAHRKHIEALLKAI |
| | HHGETEEQQKEQSRLEEELRRRLTE |
| | HEKHHQQHQEHHQHHKEHG |
| SEQ ID | SEEIHQFIQELIKQLEKEIEELVQQ |
| NO 20 | LHKELQRLRQQLQQAIERGESEEEI |
| | EQALQEFHQHFQEQLHRVRHRVHEA |
| | IKQIQQKFEKIVKDPRLREQIRQLL |
| | EQFQKQIHKALQQFQQALEEILKQI |
| | REFAKDSEDEEEAKKAHHEASRLEE |
| | ELRRRLTEEKKQHQEHHRQHQEHHQ |
| | HG |
| SEQ ID | DEEIRQIIQELIEQLRKHIHELVEQ |
| NO 21 | LHKHLRQLQEQAAQAEKRGESEEEK |
| | AKRLHQFHEQFKKHLEEVRQQVQQK |
| | IQQIQQQIARVVKDPHLRRHLHQLL |
| | QRFHQQIEEALQQFHQHLHEHLEAA |
| | KEDRSPEEHQKEESRLEEELRRRLT |
| | EEKKQHQEQHEKHEQEAQKS |
| SEQ ID | SQQIQEQLQQLIEELAQQIEHLVQQ |
| NO 22 | LAQHLHELIEQIHQLREKGKSEEEL |
| | KKHLEEFQRQFQQQLERVHHAVQKK |
| | IQQIQQHVKHLLEEHKKKSKTEEEH |
| | EHLHHLHQHIQQVLRQFAEQIQEAL |
| | KQFRKHLREALQHFEEARHSTDEEE |
| | ERKKHAEHRSRLEEELRRRLTERER |
| | QHKEQQREHHEAAQHG |
| SEQ ID | SREEVHQLHQQAHEIAQQIHQHLAH |
| NO 23 | FAEQIRRVQKQVEEHEEHPETEERQ |
| | EELKKLLKQLREQLKHLQHHVKQVE |
| | HQFKQIQRQLREHKKRGFETQELQE |
| | HLEQLEKHLQHIQQHVKALEKAVHQ |
| | AQEAIEKGTTEEHKEEHSRLEEELR |
| | RRLTEQKRQHEEAQHHHHQQQRRG |
| SEQ ID | SEHQVQQLHHEAHKIAEEIHKQLEE |
| NO 24 | IAQQIRKVQHAVAKTKDPEELQKLL |
| | EQLQKHLHELQEKVHQVQQQFHEIE |
| | QKLQEAKERGWSTEALEKHLHHLQE |
| | HLEQIRQHVEELHKAIHQAQEHIRQ |
| | HRERKKNNDERREEEQREEREKQSR |
| | LEEELRRRLTEERQHHQQQHHHHQQ |
| | QHQHG |
| SEQ ID | SEEVHQEQHKVHQEIRQHLHEVAHH |
| NO 25 | IQRHIHQFAQEVQHHREEEKDEEKR |
| | IKHIQKRLKELHEHIQQLLHKLEEH |
| | VHQLLEQLHKKVRQFQEKNPGENPE |
| | QHQHHLQQIQEQAHQAVRQLKEAAK |
| | EAQEHLHEQVEHHLRGKKDKEEHQQ |
| | RQSRLEEELRRRLTEREREQQEQAE |
| | QAHQQG |
| SEQ ID | DHELQEQLHEQLQQFEQRLAEEIER |
| NO 26 | VHRHAREAAQKGTDEEEFQQHIQQL |
| | LRKLQKHIHQVQKQIQEHLREFAQQ |
| | IKDPELQKQAKEHVHKFLQHLQEAV |
| | EKVLKHLQEHLHKHQHAKEKGEDTE |
| | EQKERRHQEESRLEEELRRRLTERE |
| | QQHREEQQQHHHHG |
| SEQ ID | DEQIQRIAHEFQQLHEKIRQLLEEL |
| NO 27 | AEELRQLAKAVHHEDKDLEQLRKEL |
| | EQLEKKIREVFKELQKLFHKLEKLL |
| | KEQPDTEELQQLQEQIEQHLQHVQQ |
| | AFKAFQKALEQLKQQLQGTTSEEQR |
| | ERQQQEASRLEEELRRRLTEHRQRH |
| | ERHHEEHQHQG |
| SEQ ID | DEEIQQHLQKLIQELAEHIKKLVQE |
| NO 28 | LQEHLHQLQQQLHAAVEKGESEEEA |
| | EERLQRFHEQFQEQLKQVHQHVAQK |
| | IKQIQQQVKQLLHSEHLHHRIQRLL |
| | EKFQEHIHRILQQFERQLKHALKQI |
| | RELLKSDDEEEERKAKQEQEREESR |
| | LEEELRRRLTEEEQQHREAQHKAHR |
| | HG |
| SEQ ID | DEELRQHIHELHQELQKQIHQLHQE |
| NO 29 | IQKQLHQLQQQIADDDGSEEERHKR |
| | LQQLLEQFKEHVHQILQEIRKAVQK |
| | ILHRIKANGEDEEHVQQAAKHVHKA |
| | VEQVEERFQQQLQHFQEHVERHREE |
| | AKTEDEEEKKQAKERHKEEHSRLEE |
| | ELRRRLTEHERQHRKHAQEHQQQG |
| SEQ ID | SHELQEHIQELHRQLQEEIQKIHEH |
| NO 30 | IQKRLEKIQHEIQQAERRGEDEEEF |
| | QQRLQQLLEQFQQHVEELLQQIKKH |
| | VEHILQRIREQVEQHKKKGID |
| | PERI |
| | EEHVQAAEKHFKHHVQQVKHAFQQH |
| | LEHFHQHVEQAKEEAKRGGNPEHAQ |
| | RAREEHSRLEEELRRRLTERQQEQH |
| | KEHQQKAHKG |
| SEQ ID | DEEEIRQIQHHLHQLVEQLAKQIHK |
| NO 31 | IQEIFHQVAQQLNKRTDPEELQKIH |
| | EQLERALQQIQEHFQRAQEHFEEIQ |
| | KRLQEHEESELLQQIEEHAQQVQKH |
| | LHEVFEAIKRIVEKFHRLLHHAKRD |
| | SEEERKKQEQHRKEQSRLEEELRRR |
| | LTEKRKHHEKHAQEQQQQG |
| SEQ ID | SQEQAHQLHERLHELHHQIHQQLEE |
| NO 32 | VAQHIKEVQQQARQLREEEEDEEKV |
| | HQHLQKLLHQLHKLLEQAHKQVHEF |
| | EKQVQHVQKQLKELREKLNQSEEQQ |
| | KHVQRAQEQIQRALHHIKRHFKHLH |
| | EAVQAFEQHLNGERSEEEHAHQARE |
| | HSRLEEELRRRLTEEERKQKKHHER |
| | QQEHG |
| SEQ ID | DEELKQQLHQFHHQLHRQLHQLHQE |
| NO 33 | LQHQAQRLAKAIQRGEVDEEHIKQH |
| | LKQQQHQIHQFLRKLQEQVEEFLKQ |
| | LQANGVNNPELQEQLEKALQHIQEQ |
| | IERALEQFAQHVHQLLKHARERKEE |
| | EEKNDDEEEEHKRRQHHREEDSRLE |
| | EELRRRLTEEEQEAQKRHKKEHQKG |
| SEQ ID | SQELRQHAHQLLQEQHQRIAQHLEE |
| NO 34 | LAHHAHQLAQEIEKHAEKEETDEEE |
| | HLQHLQELLQELQKHVEKQLQQIRK |
| | QIEEIIRHIHRHLEDEEQFEHVEQA |
| | LREQLHQAEKHVEHHLQQQRQHFHH |
| | AVKRHRDSEEQRKKQQEKESRLEEE |
| | LRRRLTEHKKQQKEKAHKQRQHG |
| SEQ ID | SQQEVQELHKRAHEIAKQIHKHLEQ |
| NO 35 | FAKQIEQVQRHVQKHEEHPETEEAR |
| | EHLKQLLHELQRRLQELQEHVKQVH |
| | HQFEEIQKQLQQHKKRGWETEELHR |
| | HLHHLQQHLEQIRQAVQQLEEAVRE |
| | AQEAIRKGTTEEHHQQHQEQSRLEE |
| | ELRRRLTEEKKRAEHHHEQQKKHG |
| SEQ ID | DEEEIKQILEQLHQQLHQAQQKIEE |
| NO 36 | AHQHLHQVHKHLQETNDPEHLRELL |
| | QQLQEQLEKLEEELRHQQKHFREVH |
| | EQFKQFREKEKDEEARQHLHQILKQ |
| | AQQFFHHLQQLFHHLQQQIHRIQHA |
| | IEAREKGERSEEEQHKQAREESRLE |
| | EELRRRLTEHKKQAEERHKKARQHG |

### Example 2: Verification of Functionalized Gadgets Mechanism

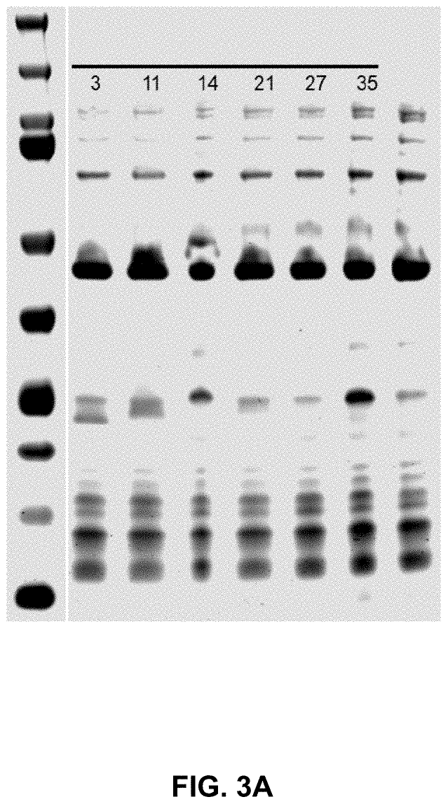

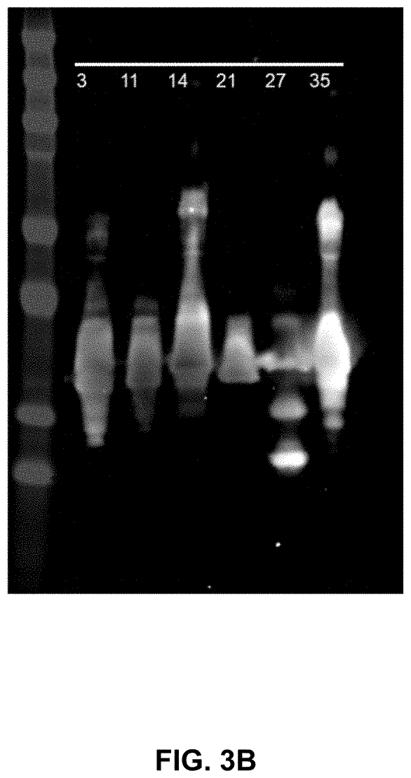

Of the above functionalized gadgets, SEQ ID Nos 3, 11, 14, 21, 27, and 35 were analyzed to determine whether these functionalized gadgets open/close relative to a standard ladder. FIGS. 3A and 3B demonstrate the opening/closing of the functionalized gadget relative to the standard ladder. This opening/closing mechanism is key to the function of the functionalized gadget. These figures are a set of standards that are used to identify the approximate size of a functionalized gadgets (in an open or closed configuration) on a gel during electrophoresis, using the principle that molecular weight is inversely proportional to migration rate through a gel matrix.

What is claimed is:

1. A bi-stable functionalized organic molecule (functionalized gadget), the bi-stable functionalized organic molecule comprises:
(a) two structurally rigid, constant regions (constant region 1 and constant region 2);
(b) two modifiable, variable regions (variable region 1 and variable region 2);
(c) a purification tag;
(d) a barcode region; and
wherein variable region 1 is covalently attached to an interior position or a terminal position of constant region 2, constant region 2 is covalently attached to an interior position or a terminal position of constant region 1, variable region 2 is covalently attached to an interior position or a terminal position of constant region 1, the purification tag is attached to an interior position of constant region 2; and the barcode region is covalently attached to an interior position or a terminal position of variable region 1 or variable region 2;
wherein the variable region 1 and variable region 2 interact through intermolecular hydrogen bonding, zwitterionic bonding, or a combination of intermolecular hydrogen bonding and zwitterionic bonding.

2. The bi-stable functionalized organic molecule of claim 1, wherein variable region 1 or variable region 2 of the bi-stable functionalized organic molecule interacts with a target analyte and undergoes a conformational change.

3. The bi-stable functionalized organic molecule of claim 2, wherein the interaction removes a portion of the intermolecular hydrogen bonding, zwitterionic bonding, or a combination of the intermolecular hydrogen bonding and zwitterionic bonding in the bi-stable functionalized organic molecule and reveals the purification tag.

4. The bi-stable functionalized organic molecule of claim 1, wherein the barcode region comprises a nucleic acid.

5. The bi-stable functionalized organic molecule of claim 4, wherein the nucleic acid comprise 20 base pairs of nucleotides to about 5000 base pairs of nucleotides.

6. The bi-stable functionalized organic molecule of claim 4, wherein the nucleic acid comprise natural nucleotides, unnatural nucleotides, synthetic nucleotides, or combinations thereof.

7. The bi-stable functionalized organic molecule of claim 4, wherein the nucleic acid comprises DNA, RNA, or a combination of DNA and RNA.

8. The bi-stable functionalized organic molecule of claim 6, wherein the nucleic acid comprises RNA.

9. The bi-stable functionalized organic molecule of claim 1, wherein variable region 1 and variable region 2 comprise a structured set of peptides, an unstructured set of peptides, or a combination of a structured set of peptides and an unstructured set of peptides.

10. The bi-stable functionalized organic molecule of claim 9, wherein the structured set of peptides or unstructured set of peptides comprise from about 3 amino acids to about 100 amino acids.

11. The bi-stable functionalized organic molecule of claim 10, wherein the amino acids comprise L-amino acids, D-amino acids, nonstandard unnatural amino acids, amino acid analogs, or a combination thereof.

12. The bi-stable functionalized organic molecule of claim 1, wherein constant region 1 and constant region 2 comprise sequences of amino acids.

13. The bi-stable functionalized organic molecule of claim 12, wherein the constant region 1 and constant region 2 comprise from about 3 amino acids to about 200 amino acids.

14. The bi-stable functionalized organic molecule of claim 13, wherein the amino acids comprise L-amino acids, D-amino acids, nonstandard unnatural amino acids, amino acid analogs, or a combination thereof.

15. The bi-stable functionalized organic molecule of claim 12, wherein the constant region 1, constant region 2, or combinations of constant region 1 and constant region 2 further comprise sites for covalent attachments.

16. The bi-stable functionalized organic molecule of claim 1, wherein the purification tag is a protein tag or a peptide tag.

17. The bi-stable functionalized organic molecule of claim 14, wherein the purification tag may comprise a single site or multiple sites for external covalent attachments.

18. The bi-stable functionalized organic molecule of claim 1, wherein the bi-stable functionalized organic molecule is an amino acid sequence from SEQ ID NOs 1 to 36.

19. The bi-stable functionalized organic molecule of claim 16, wherein the bi-stable functionalized organic molecule is an amino acid sequence from SEQ ID NOs 3, 11, 14, 21, 27, or 35.

20. The bi-stable functionalized organic molecule of claim 2, wherein the target analyte comprises a protein, a small molecule, a metabolite, or a combination of two or more thereof.


SEQUENCE LISTING (36 sequences)

SEQ ID NO: 1 - protein, 184 aa, Artificial Sequence
  SYNTHESIZED
DQEIHQHLQHLQQQLHERIKHLVQKLHEHLQHLFKQLQQAEKRGESEEERKKRLQQFRHEIQEQLQQVHEHVQKQIEEIRQQIKKLLQEARKRSKTEEEHERLKELHKQIHEALEHFHQAVQHFLEAFHKAFQEVLEAIKNGESEEEAQQRRSRLEEELRRRLTEEEKRHQQHQEEQHHAHQQG

SEQ ID NO: 2 - protein, 175 aa, Artificial Sequence
  SYNTHESIZED
DEEARKILQQLREQAERAHKHVQEVHQQFQEIQERIRHLEERNDENVEEHLKHFERQIKHLFQQIQQIFQELQEHLKEAKEHAKKNNHSPHLKEAIEQIEQQVHQLFKQIQQIVEHLKQLLHQAKQAHENPEDERRKEQKEEASRLEEELRRRLTEHEQRQHEQHEQQKQRHQHG

SEQ ID NO: 3 - protein, 175 aa, Artificial Sequence
  SYNTHESIZED
SEELQQQIQELAKKAQEVHQHIHKLLEEFHQLLEELRHSTDSPEELKKKLEELHQQLQKVLQKAQQLFRQLHELFRRLQEAAEKENISPEQKQHQEQQLRAVEHHFRAAQRFFKHFQQLLHQLHEHQEKERSEEERKHHEKEASRLEEELRRRLTEREQEHRQQQKQHQEHQQHG

SEQ ID NO: 4 - protein, 176 aa, Artificial Sequence
  SYNTHESIZED
DEELQKRIHELAHQLQRQIHHIQHHIHEELKQIHEEIQQHEEKGRDKEEHKQRLHQLLHQFRQHVQELLQQIREQVQQILQHIQEKGTNEEHVKAAEQAFKHAVEQVEQAFEQQLKQFAQHVKQAEEHSDRDEEEREKHRKQAKKERSRLEEELRRRLTEERQHHHEEHHHQQQKG

SEQ ID NO: 5 - protein, 172 aa, Artificial Sequence
  SYNTHESIZED
SQEIAEELQKLAQELEHHIEELVKQLQKQLEHLAQQAEQAKKRGESEEEREERLQRFQQHIHEQLKQFHQHVHEKIHQIQQHVRHLLEKHNESSELEHHIQELLQQFHEHFHQALQHFQEEVQKQLEEARRDKKSPEQRQEERSRLEEELRRRLTEHKKAQKKHRHAQQQRG

SEQ ID NO: 6 - protein, 176 aa, Artificial Sequence
  SYNTHESIZED
SQEILHELQKKFHHVQEQIRQLAKEIQQILHEAQQLLHSDRSEEEIEEIHRQLKELLKQVHQQLRKIEQLVRQIEKQIQELRERGTHSEEQHEHIEQLLQELQKALKHLQEHVRKAHEQFQKILEALQAARRNETDPEEKHKQHREASRLEEELRRRLTEEEKQQQEQHQKAQQHG

SEQ ID NO: 7 - protein, 179 aa, Artificial Sequence
  SYNTHESIZED
DEELHQAIQELQQQLHKQIQKLAQQIHKQLHEIAQEIHELAERGRDEEEIKRRLKELLEHFEQHVREILQEIQQQAQRILHKIRASGESEEQVQAAHHHIQRAVEHVKKHFHKQLEQFRKHVEQHQKEEEDSNTSKEEEHKRQKQHQQEESRLEEELRRRLTEDEKRHHEHHQQQHQQG

SEQ ID NO: 8 - protein, 168 aa, Artificial Sequence
  SYNTHESIZED
DEEEIHEIQHRLEQLIHELAKHFRHFAHHFHEVQQQLNKDTSEEQLKQIEEQLQEHHKQLEKHFRQAQKEFRKIQQHLQQLRESEQLRKIHKQVQHALEHAEHVFEQIRQIVEQFEEQLHAAKRGKHDEEKAKKQHEERSRLEEELRRRLTEEEQQQQQHQHEHQRHG

SEQ ID NO: 9 - protein, 175 aa, Artificial Sequence
  SYNTHESIZED
DQELLHQLQEKVHQVQQQIRELAQQIERLLQRIHKLLQSDRSEEEIQHIAQQLREFLQQVHKQLQEIAEQVEKIAQEAEEIKKRGVDEEQRKAIQQLVEHLHKHLERLQKHVHQAHEAFERILKQLEHREKKGKPSEDHEEHHEEESRLEEELRRRLTEEEKQAQKQHQQHQRQG

SEQ ID NO: 10 - protein, 168 aa, Artificial Sequence
  SYNTHESIZED
SKEELQELRQQAHQHQKQLHELLHKIHEQLHQHAHEIKDKDPHHLQRIQQHLQELLEHIHHIVREFQQHVKEVAQQLREAQEKEKDEEEQRRLEEHLQHLQHALKRFKQHFEQAHEHVQQLQHAEKQSEEEHKKQHEERSRLEEELRRRLTEHKQHHEEHERRAQQHG

SEQ ID NO: 11 - protein, 175 aa, Artificial Sequence
  SYNTHESIZED
DHELKEKIHHLQQQLRERIEELHRQIAEQLEQIAQQIHSDDGSEEEAAKRLQHLLHQFQEHVKQILQEIQQHIHEILHQIKASGTDEHQVQQAEEHFEQAVRQVERHFQQQLKRFEEHVQRAKEEREKNRSEEEKEQAKQHEQEKHSRLEEELRRRLTEEKKKQHQHHQQHRKQG

SEQ ID NO: 12 - protein, 164 aa, Artificial Sequence
  SYNTHESIZED
DHEQVHQLQQQAHEIREEIHQQLKRIAQKIREVQQHVEEHKKHPETEEQRQHLEQLLRELHHQLERLKQHVKQVEKQFQEIQKHETDEEHLRELQEALKQIQEAVHHLEHAIHRAQQHIKDKHSEEEHQREKSRLEEELRRRLTEHEQQQEQAKKAHRQHQHHG

SEQ ID NO: 13 - protein, 170 aa, Artificial Sequence
  SYNTHESIZED
SQEELQALQQQAQHHARKIQEKLEEIHERLRQFAEQIKDKDPHELHKIIQQLQELLEELQRQVEEFEKHVRHVARQLKKHAKEEKDEERRQQLEKHLKQLQKHLKQAQEEFHQHEHLIRQLIAQLQSEEDNPEEEHEHSRLEEELRRRLTEHKQQHQRHAEQHQQQHRHG

SEQ ID NO: 14 - protein, 185 aa, Artificial Sequence
  SYNTHESIZED
SHEEVHQLHREAHQIQRQIQEQLHQIHQHIHEVHQAVKKAQESEDTEEAQQHLQQLLKQLQEQLERLKHHVQKVEQAFHRIQRHLEEHERKINGSEEHRKQLQKHLKHLREALEQIKRHVKQLQEAIQRQEEHIKKAKDDSSEEERHQEQQEESRLEEELRRRLTEEREQHEQQHHQAHKEHKHG

SEQ ID NO: 15 - protein, 174 aa, Artificial Sequence
  SYNTHESIZED
DEELHKAIKKLHEQAQQVHRAIHEFQKQIEELLEQLQKKEPSEELLQELKQLEKQLHEILQQAQRLFEQLHKLFQELEEEAKKRPESEEHQQAAQQRREALEEVRKHFEHVQKAFQHFQHVLQHLQHQLRHNDGNEEQQKRESRLEEELRRRLTERKQRHHRHHEQHEQQHQHG

SEQ ID NO: 16 - protein, 164 aa, Artificial Sequence
  SYNTHESIZED
SEEIHHILQRLIEQLAKQIKELVEQLQEQLRHLQQRAQKAEERGKSEEEKQQQLEEFHKQLEKALERVHQRVAQQIQHIQQHIAQIVKDPELRKHIEHLLEAFKQHVEQFLEEFHQRIRHHLQHPETSDEEESRLEEELRRRLTEKEQQQQHHQEEARKQQKKG

SEQ ID NO: 17 - protein, 178 aa, Artificial Sequence
  SYNTHESIZED
DEELQEKIHQLQHQLHHKIQQIQEHIRQQLHQIQEEIQQAIEKGRDEEEHIERLRQLLEHFRQQVHQLLQQIEQQVKRILHHIKANGEDEHHVQQAQEHFEQQVKQVKEHFQAALKQFEEHVKKAIEEFKSEDSEEEKKKRHQHERSRLEEELRRRLTEHRRQQKEAAEEARQHHHEG

SEQ ID NO: 18 - protein, 178 aa, Artificial Sequence
  SYNTHESIZED
DEELKQRIHELHQQAEQVHRQIHQVFKKFHELLKELQAQLKEHGTETEELRQKLQELHEELKRVLEEAQKLFRELHQLFRELQQQAKENNESEEEQEHQHRALQHVQEHVKQAQQFFKHFQRLLHALQEIAKGTSEEEAKRRQQKHSRLEEELRRRLTEHKQQHEEQQQHHEQHHRQG

SEQ ID NO: 19 - protein, 169 aa, Artificial Sequence
  SYNTHESIZED
DEEELRQLEHHLHQAQQELHEILQQIQEHLEQAAQLIKDKDPEELKKILQKLRQLLRRLRHQVQRFEQLVKHVEERLKEFHKREKDEERQKQLQEHLQQLQEHLRQAQEAFEAHRKHIEALLKAIHHGETEEQQKEQSRLEEELRRRLTEHEKHHQQHQEHHQHHKEHG

SEQ ID NO: 20 - protein, 177 aa, Artificial Sequence
  SYNTHESIZED
SEEIHQFIQELIKQLEKEIEELVQQLHKELQRLRQQLQQAIERGESEEEIEQALQEFHQHFQEQLHRVRHRVHEAIKQIQQKFEKIVKDPRLREQIRQLLEQFQKQIHKALQQFQQALEEILKQIREFAKDSEDEEEAKKAHHEASRLEEELRRRLTEEKKQHQEHHRQHQEHHQHG

SEQ ID NO: 21 - protein, 170 aa, Artificial Sequence
  SYNTHESIZED
DEEIRQIIQELIEQLRKHIHELVEQLHKHLRQLQEQAAQAEKRGESEEEKAKRLHQFHEQFKKHLEEVRQQVQQKIQQIQQQIARVVKDPHLRRHLHQLLQRFHQQIEEALQQFHQHLHEHLEAAKEDRSPEEHQKEESRLEEELRRRLTEEKKQHQEQHEKHEQEAQKS

SEQ ID NO: 22 - protein, 191 aa, Artificial Sequence
  SYNTHESIZED
SQQIQEQLQQLIEELAQQIEHLVQQLAQHLHELIEQIHQLREKGKSEEELKKHLEEFQRQFQQQLERVHHAVQKKIQQIQQHVKHLLEEHKKKSKTEEEHEHLHHLHQHIQQVLRQFAEQIQEALKQFRKHLREALQHFEEARHSTDEEEERKKHAEHRSRLEEELRRRLTERERQHKEQQREHHEAAQHG

SEQ ID NO: 23 - protein, 174 aa, Artificial Sequence
  SYNTHESIZED
SREEVHQLHQQAHEIAQQIHQHLAHFAEQIRRVQKQVEEHEEHPETEERQEELKKLLKQLREQLKHLQHHVKQVEHQFKQIQRQLREHKKRGFETQELQEHLEQLEKHLQHIQQHVKALEKAVHQAQEAIEKGTTEEHKEEHSRLEEELRRRLTEQKRQHEEAQHHHHQQQRRG

SEQ ID NO: 24 - protein, 180 aa, Artificial Sequence
  SYNTHESIZED
SEHQVQQLHHEAHKIAEEIHKQLEEIAQQIRKVQHAVAKTKDPEELQKLLEQLQKHLHELQEKVHQVQQQFHEIEQKLQEAKERGWSTEALEKHLHHLQEHLEQIRQHVEELHKAIHQAQEHIRQHRERKKNNDERREEEQREEREKQSRLEEELRRRLTEERQHHQQQHHHHQQQHQHG

SEQ ID NO: 25 - protein, 181 aa, Artificial Sequence
  SYNTHESIZED
SEEVHQEQHKVHQEIRQHLHEVAHHIQRHIHQFAQEVQHHREEEKDEEKRIKHIQKRLKELHEHIQQLLHKLEEHVHQLLEQLHKKVRQFQEKNPGENPEQHQHHLQQIQEQAHQAVRQLKEAAKEAQEHLHEQVEHHLRGKKDKEEHQQRQSRLEEELRRRLTEREREQQEQAEQAHQQG

SEQ ID NO: 26 - protein, 164 aa, Artificial Sequence
  SYNTHESIZED
DHELQEQLHEQLQQFEQRLAEEIERVHRHAREAAQKGTDEEEFQQHIQQLLRKLQKHIHQVQKQIQEHLREFAQQIKDPELQKQAKEHVHKFLQHLQEAVEKVLKHLQEHLHKHQHAKEKGEDTEEQKERRHQEESRLEEELRRRLTEREQQHREEQQQHHHHG

SEQ ID NO: 27 - protein, 161 aa, Artificial Sequence
  SYNTHESIZED
DEQIQRIAHEFQQLHEKIRQLLEELAEELRQLAKAVHHEDKDLEQLRKELEQLEKKIREVFKELQKLFHKLEKLLKEQPDTEELQQLQEQIEQHLQHVQQAFKAFQKALEQLKQQLQGTTSEEQRERQQQEASRLEEELRRRLTEHRQRHERHHEEHQHQG

SEQ ID NO: 28 - protein, 177 aa, Artificial Sequence
  SYNTHESIZED
DEEIQQHLQKLIQELAEHIKKLVQELQEHLHQLQQQLHAAVEKGESEEEAEERLQRFHEQFQEQLKQVHQHVAQKIKQIQQQVKQLLHSEHLHHRIQRLLEKFQEHIHRILQQFERQLKHALKQIRELLKSDDEEEERKAKQEQEREESRLEEELRRRLTEEEQQHREAQHKAHRHG

SEQ ID NO: 29 - protein, 174 aa, Artificial Sequence
  SYNTHESIZED
DEELRQHIHELHQELQKQIHQLHQEIQKQLHQLQQQIADDDGSEEERHKRLQQLLEQFKEHVHQILQEIRKAVQKILHRIKANGEDEEHVQQAAKHVHKAVEQVEERFQQQLQHFQEHVERHREEAKTEDEEEKKQAKERHKEEHSRLEEELRRRLTEHERQHRKHAQEHQQQG

SEQ ID NO: 30 - protein, 185 aa, Artificial Sequence
  SYNTHESIZED
SHELQEHIQELHRQLQEEIQKIHEHIQKRLEKIQHEIQQAERRGEDEEEFQQRLQQLLEQFQQHVEELLQQIKKHVEHILQRIREQVEQHKKKGIDPERIEEHVQAAEKHFKHHVQQVKHAFQQHLEHFHQHVEQAKEEAKRGGNPEHAQRAREEHSRLEEELRRRLTERQQEQHKEHQQKAHKG

SEQ ID NO: 31 - protein, 169 aa, Artificial Sequence
  SYNTHESIZED
DEEEIRQIQHHLHQLVEQLAKQIHKIQEIFHQVAQQLNKRTDPEELQKIHEQLERALQQIQEHFQRAQEHFEEIQKRLQEHEESELLQQIEEHAQQVQKHLHEVFEAIKRIVEKFHRLLHHAKRDSEEERKKQEQHRKEQSRLEEELRRRLTEKRKHHEKHAQEQQQQG

SEQ ID NO: 32 - protein, 180 aa, Artificial Sequence
  SYNTHESIZED
SQEQAHQLHERLHELHHQIHQQLEEVAQHIKEVQQQARQLREEEEDEEKVHQHLQKLLHQLHKLLEQAHKQVHEFEKQVQHVQKQLKELREKLNQSEEQQKHVQRAQEQIQRALHHIKRHFKHLHEAVQAFEQHLNGERSEEEHAHQAREHSRLEEELRRRLTEEERKQKKHHERQQEHG

SEQ ID NO: 33 - protein, 175 aa, Artificial Sequence
  SYNTHESIZED
DEELKQQLHQFHHQLHRQLHQLHQELQHQAQRLAKAIQRGEVDEEHIKQHLKQQQHQIHQFLRKLQEQVEEFLKQLQANGVNNPELQEQLEKALQHIQEQIERALEQFAQHVHQLLKHARERKEEEEKNDDEEEEHKRRQHHREEDSRLEEELRRRLTEEEQEAQKRHKKEHQKG

SEQ ID NO: 34 - protein, 173 aa, Artificial Sequence
  SYNTHESIZED
SQELRQHAHQLLQEQHQRIAQHLEELAHHAHQLAQEIEKHAEKEETDEEEHLQHLQELLQELQKHVEKQLQQIRKQIEEIIRHIHRHLEDEEQFEHVEQALREQLHQAEKHVEHHLQQQRQHFHHAVKRHRDSEEQRKKQQEKESRLEEELRRRLTEHKKQQKEKAHKQRQHG

SEQ ID NO: 35 - protein, 174 aa, Artificial Sequence
  SYNTHESIZED
SQQEVQELHKRAHEIAKQIHKHLEQFAKQIEQVQRHVQKHEEHPETEEAREHLKQLLHELQRRLQELQEHVKQVHHQFEEIQKQLQQHKKRGWETEELHRHLHHLQQHLEQIRQAVQQLEEAVREAQEAIRKGTTEEHHQQHQEQSRLEEELRRRLTEEKKRAEHHHEQQKKHG

SEQ ID NO: 36 - protein, 175 aa, Artificial Sequence
  SYNTHESIZED
DEEEIKQILEQLHQQLHQAQQKIEEAHQHLHQVHKHLQETNDPEHLRELLQQLQEQLEKLEEELRHQQKHFREVHEQFKQFREKEKDEEARQHLHQILKQAQQFFHHLQQLFHHLQQQIHRIQHAIEAREKGERSEEEQHKQAREESRLEEELRRRLTEHKKQAEERHKKARQHG